(19) United States
(12) Patent Application Publication
(10) Pub. No.: US 2025/0144357 A1
(43) Pub. Date: May 8, 2025

(54) FLEXIBLE FLUID GUIDE UNIT WITH HIGH BUCKLING STABILITY

(71) Applicant: Drägerwerk AG & Co. KGaA, Lübeck (DE)

(72) Inventors: Jöran BLENDERMANN, Lübeck (DE); Claas-Christian Reiner SEELHOFF, Lübeck (DE)

(21) Appl. No.: 18/938,835

(22) Filed: Nov. 6, 2024

(30) Foreign Application Priority Data
Nov. 8, 2023 (DE) 10 2023 130 909.9

(51) Int. Cl. A61M 16/10; A61M 16/08

(52) U.S. Cl. CPC A61M 16/1095; A61M 16/0875; A61M 2205/3653

(57) ABSTRACT

A flexible fluid guide unit (32) includes an inner tube (10), an outer tube (12), a spiral coil (11) between the inner tube (10) and the outer tube and a heating element (15.1, 15.2). The heating element heats the fluid carried by the inner tube. The coil extends around the inner tube. An outer surface of the outer tube comes into thermal contact with an environment and is smaller than the outer surface of the coil plus an uncovered part of an outer surface of the inner tube. The coil Shore hardness is at least 10 shoreA greater than the inner tube and outer tube Shore hardness. Or the coil is hollow and a wall of the coil has a wall thickness that is at least twice as great as the wall thickness of the inner tube and at least twice as great as that of the outer tube.

### CROSS REFERENCE TO RELATED APPLICATIONS

This application claims the benefit of priority under 35 U.S.C. § 119 of German Application 10 2023 130 909.9, filed Nov. 8, 2023, the entire contents of which are incorporated herein by reference.

### TECHNICAL FIELD

The invention relates to a flexible fluid guide unit that can be used in an application for artificial ventilation (artificial respiration) of a patient.

### BACKGROUND

In the following, a fluid guide unit is understood to be a component that is able to guide a fluid, e.g. a gas, along a trajectory, whereby this trajectory is predetermined by the design and arrangement of the fluid guide unit. Ideally, the fluid guide unit prevents the fluid flowing through the fluid guide unit from leaving this trajectory.

A “flexible component” (pliable component) is a component that extends along a longitudinal axis and can reversibly be bent about an axis that is perpendicular (orthogonal) to the longitudinal axis. A flexible component therefore suffers no significant damage due to bending-provided that the radius of curvature remains above a lower threshold specified by the design and material of the component.

A flexible fluid guide unit is a fluid guide unit which, even when bent around an axis being perpendicular to the longitudinal axis, is still able to guide a sufficient amount of fluid along the trajectory. In particular, the available cross-sectional area is not reduced very significantly. A flexible fluid guide unit is in particular a suitably constructed tube (hose).

Such a flexible fluid guide unit is used, for example, for artificial ventilation of a patient. The patient is connected to a patient-side coupling unit, in particular to a tube (e.g. an endotracheal tube) or a breathing mask. A ventilator performs a sequence of ventilation strokes and expels (emits/ejects) in each ventilation stroke a respective quantity of a breathable gas mixture, whereby the gas mixture comprises oxygen. The breathable gas mixture may additionally comprise at least one anesthetic agent and/or a drug. The quantity of the gas mixture expelled in a ventilation stroke is guided through the flexible fluid guide unit to the patient-side coupling unit so that the patient can inhale the quantity. It is possible that a ventilation circuit is established and the gas mixture exhaled by the patient is returned to the ventilator. In order to return the exhaled gas mixture, a further fluid guide unit is used, preferably a further flexible fluid guide unit.

### SUMMARY

It is an object of the invention to provide a flexible fluid guide unit in which the operational safety is increased in comparison with known flexible fluid guide units.

The problem is solved by a flexible fluid guide unit with flexible fluid guide unit features according to the invention. Advantageous embodiments of the fluid guide unit according to the invention are disclosed.

According to the invention, an inner tube of the fluid guide unit is configured to guide a fluid along a trajectory, the trajectory being predetermined by the configuration and the arrangement and positioning of the fluid guide unit. Preferably, the inner tube has a smooth inner surface over its entire length, i.e. in particular it is not a corrugated tube. This reduces the risk of turbulence occurring during the flow of the fluid. Preferably, the inner tube also has a smooth outer surface over its entire length.

A heating element of the fluid guide unit is able to heat a fluid while this fluid is being guided by the inner tube. The heating element can thereby cause the temperature of the fluid to be higher than the ambient temperature and/or prevent the initially warmer fluid from cooling down to the ambient temperature as it flows through the inner tube. For example, the heating element keeps the breathable gas mixture at a temperature that deviates from the patient's body temperature by not more than a predetermined tolerance. Preferably, the heating element is in thermal contact with a fluid flowing through the inner tube.

In one embodiment, the heating element is embedded in the inner tube; in another embodiment, the heating element is embedded in the coil described below. It is also within the scope of the invention that the heating element heats the inner tube without contact, for example with the aid of a radiation source that emits electromagnetic radiation in the direction of the inner tube, in particular infrared radiation.

The inner tube extends along a longitudinal axis and is preferably rotationally symmetrical to this longitudinal axis. A spiral coil (spiral helix) is applied to the outer surface of the inner tube. The coil is guided in a spiral around the inner tube. In other words, the coil has the shape of a hollow rod (hollow bar/hollow strand) or filled rod (non-hollow bar/non-hollow strand) that is formed into the shape of a spiral. An outer tube is attached to the coil. The outer tube therefore surrounds the coil and the inner tube. The coil is mechanically connected to both the inner tube and the outer tube. The coil thus covers a spiral-shaped part of the outer surface of the inner tube, while another part of the outer surface of the inner tube is not covered by the coil. Preferably, the coil also covers a spiral-shaped part of the inner surface of the outer tube. A spiral-shaped cavity occurs between the coil and the outer tube.

The outer surface, i.e. outward-facing surface of the outer tube, has a smaller area than the following sum (the sum of):
the area of the outer surface of the spiral coil; and
the area of that part of the outer surface of the inner tube that is not covered by the coil.

The outer surface of the outer tube or at least one segment of the outer tube is in thermal contact with an environment of the fluid guide unit, in one application in thermal contact with ambient air.

The inner tube, the outer tube and the coil each have a Shore hardness, whereby the Shore hardness is measured in shoreA (Shore A hardness scale). The three Shore hardnesses can be the same or different. The Shore hardness of the coil is at least as high as the Shore hardness of the inner tube and at least as high as the Shore hardness of the outer tube.

The invention specifies three different alternatives, whereby according to the invention at least one alternative is fulfilled and at least two of these alternatives can be implemented in combination.

According to the first alternative, the Shore hardness of the coil is at least 10 shoreA greater than the Shore hardness of the inner tube. In addition, the Shore hardness of the coil is at least 10 shoreA greater than the Shore hardness of the outer tube. Preferably, the Shore hardness of the coil is at least 20 shoreA, particularly preferably at least 30 shoreA, especially at least 40 shoreA, greater than the Shore hardness of the inner tube. Preferably, the Shore hardness of the coil is at least 20 shoreA, particularly preferably at least 30 shoreA, especially at least 40 shoreA, greater than the Shore hardness of the outer tube. According to the first alternative the coil can be hollow or filled (non-hollow).

According to a second alternative, the coil is hollow. In other words, the coil has the shape of a hollow rod that is guided in a spiral around the inner tube. The coil has a wall. The wall of the coil surrounds a spiral-shaped cavity. The wall of the spiral coil has a wall thickness. The inner tube and the outer tube also each have a wall with a wall thickness. According to the second alternative, the wall thickness of the spiral coil is at least twice as great as the wall thickness of the inner tube and at least twice as great as the wall thickness of the outer tube.

According to a third alternative, the coil is filled, i.e. it does not have a spiral-shaped cavity. In other words, the coil has the shape of a filled rod or stick that is spirally wound around the inner tube. The maximum dimension of this spiral-shaped rod in a direction parallel to the longitudinal axis of the inner tube is at least four times as great as the wall thickness of the inner tube, i.e. at least four times as great as the minimum dimension of the wall of the inner tube in a direction perpendicular to the longitudinal axis. In addition, the maximum dimension of the spiral-shaped rod in the parallel direction is at least four times as great as the wall thickness of the outer tube.

According to the invention, the heating element is able to heat a fluid which is guided by the inner tube. As a result, the fluid can permanently have a higher temperature than the ambient temperature while the fluid flows through the inner tube. Preferably, the heating element causes the temperature of the fluid to remain within a predetermined temperature range along the entire longitudinal axis of the inner tube and during use of the fluid guide unit.

This effect is important, for example, when artificially ventilating a patient. The fluid guide unit according to the invention bridges a distance between a ventilator and a patient-side coupling unit or at least contributes to bridging this distance. The patient-side coupling unit is arranged in and/or on the patient's body. The patient-side coupling unit comprises, for example, a breathing mask and/or a tube. The ventilator expels a breathable gas mixture, and the gas mixture is supplied to the patient-side coupling unit and thereby to the patient through the fluid guide unit according to the invention. The gas mixture should generally have a temperature which lies within a predetermined temperature range, this temperature preferably being approximately equal to the body temperature of a human being and generally being greater than the ambient temperature. As a rule, the lower threshold of the temperature range is therefore greater than the temperature in the vicinity of the fluid guide unit.

As already described above, the fluid guide unit according to the invention is able to bring about the higher temperature through the heating element. If the temperature of the gas mixture is higher than the ambient temperature, the fluid guide unit inevitably transfers thermal energy to the environment. As a result, a fluid in the fluid guide unit is also cooled and the heating element must compensate for the cooling.

According to the invention, the outer tube has a smaller outer surface area than the coil plus the part of the inner tube that is not covered by the coil. This uncovered part is also spiral-shaped. It is known that—for a given temperature difference and a given material—the larger the surface area is, the greater is the amount of heat that is dissipated through a surface. The outer tube therefore reduces the loss of thermal energy compared to a configuration in which the spiral coil and the uncovered part of the inner tube come into direct thermal contact with the environment.

The space between the inner tube and the outer tube also helps to thermally insulate a fluid in the inner tube from the environment to a certain degree.

In many cases, it is not possible to prevent the fluid guide unit from buckling, being kinked or bent, especially if it is guided around a rigid object and, as a result, the flow direction of a fluid changes significantly, in particular by at least 90 degrees. The following danger can occur in this situation: The cross-sectional area available for the fluid flowing through can be considerably reduced in the buckled, kinked or bent area compared to the rest of the fluid guide unit. In many cases, this also reduces the achievable volume flow, i.e. the volume per unit of time, through the fluid guide unit. If the fluid guide unit is used for artificial ventilation of a patient, this can put the patient at risk. If the kink, bend or buckle is suddenly removed, the likewise often undesirable event can occur that a surge of a gas mixture reaches the patient-side coupling unit and thus the patient, causing a bolus.

The invention offers a compromise between the following two contradicting requirements:
The fluid guide unit should be sufficiently flexible, especially in order to be able to adapt it to given spatial boundary conditions.
Even if the fluid guide unit is buckled or kinked or bent, the volume flow through the inner tube should not be reduced too much.

In order to use a fluid guide unit, it is often necessary to bend the fluid guide unit. In internal tests, the inventors have found that the buckled or kinked or bent fluid guide unit retains (maintains) a sufficiently large cross-sectional area if the fluid guide unit has a sufficiently large buckling stability (kink resistance). In many cases, all three alternatives of the invention result in the flexible fluid guide unit having a sufficiently large buckling stability. In many cases, all three alternatives have the following effect when the fluid guide unit according to the invention is bent or kinked or becomes buckled around a rigid object: A segment of the inner tube and a segment of the outer tube pointing away from the object are stretched, i.e. pulled apart. With conventional flexible fluid guide units, on the other hand, a segment of the inner tube pointing towards the object is pressed against an opposite segment, i.e. against the outer tube, which reduces the distance between these two segments and thus the cross-sectional area.

In internal tests, the inventors have found that a fluid guide unit according to the invention generally fulfills the following requirements for buckling stability, whereby these requirements were derived from the standard EN ISO 5367:2023. Sufficiently high buckling stability means that the pneumatic resistance of the fluid guide unit does not increase significantly when the fluid guide unit is bent or kinked. The inventors have used the following procedure to check whether this requirement for buckling stability is met:

The pneumatic resistance of the fluid guide unit at a given reference volume flow of a gas through the fluid guide unit and at given reference properties of the gas is measured twice. As is known, the pneumatic resistance of a fluid guide unit is the quotient of the pressure drop (pressure loss) at the fluid guide unit and the volume flow through the fluid guide unit. In both measurements, a predetermined (given) reference volume flow of a gas is achieved through the fluid guide unit, whereby the gas has predetermined reference properties. The reference volume flow is 30 l/min for adults, 15 l/min for adolescents and children and 2.5 l/min for newborns. The reference properties of the gas are: temperature between 39 and 45 degrees C., relative humidity at least 80%. These are typical reference properties of a breathable gas mixture that is delivered to a patient during artificial ventilation.

During the first measurement, the fluid guide unit is not buckled or kinked or bent, but has the shape of a straight rod. The fluid guide unit is then bent around a rigid rod with a round cross-sectional area, wherein the rod has a diameter of 2.5 cm and wherein the wrap angle of the fluid guide unit around the rod is at least 180°. In other words, the bent (curved) fluid guide unit surrounds at least half of the circumference of the rigid rod. In the second measurement, the pneumatic resistance of the fluid guide unit bent (curved) as just described is measured.

The fluid guide unit has sufficient buckling stability if the pneumatic resistance in the second measurement, i.e. in the buckled fluid guide unit, is at most 50% greater than the pneumatic resistance in the first measurement. A practical background: In many cases, the pressure at which a breathable gas mixture flows through a fluid guide unit is regulated during artificial ventilation of a patient. As a rule, the event that the volume flow is reduced due to a kink is not fully or at least not immediately compensated for by an increase in pressure. If the fluid guide unit has a sufficiently high buckling stability, the volume flow through the fluid guide unit is still sufficiently high, even if the fluid guide unit is bent, which can occur frequently in practice as already explained.

The invention thus produces two effects, both of which are particularly important in the artificial ventilation of a patient: on the one hand, good thermal insulation of the fluid in the inner tube from the environment and, on the other hand, sufficiently high buckling stability. The invention achieves these two effects by means of two different components, namely the thermal insulation provided by the outer tube and the high buckling stability provided by the coil, which is configured in accordance with at least one of the three alternatives. The invention does not require a single component of the fluid guide unit to be optimized with regard to both requirements, which is often not possible in practice.

As a rule, both the outer tube and the inner tube have a circular cross-sectional area, at least if they are not kinked or bent. Preferably, the outer diameter of the outer tube is 8 cm or less. This design helps to ensure that the fluid guide unit is sufficiently flexible. The outer diameter of the outer tube is preferably between 1 cm and 5 cm, in particular between 20 mm and 30 mm. The outer diameter of the inner tube is preferably at least 5 mm smaller than the inner diameter of the outer tube. In many cases, this configuration ensures that a sufficient amount of fluid can be guided through the inner tube, provided that the fluid guide unit has the sufficiently high buckling stability as described above.

The buckling stability of a tubular component is essentially determined by the Shore hardness and also by the wall thickness. The Shore hardness is an indicator of the stiffness of a flexible component and is measured in shoreA according to the invention. In a preferred embodiment, the Shore hardness of the coil is at least 30% greater than the Shore hardness of the inner tube, preferably at least 50% greater. Particularly preferably, the Shore hardness of the coil is also at least 30% greater than the Shore hardness of the outer tube, preferably at least 50% greater. This design often leads to particularly good buckling stability.

A preferred embodiment of the invention specifies value ranges for the Shore hardnesses. Preferably, the Shore hardness of the coil is at least 70 shoreA. Preferably, the Shore hardness of the coil is below 90 shoreA, particularly preferably below 80 shoreA. In one embodiment, the Shore hardness of the coil is between 75 shoreA and 85 shoreA. This design often leads to particularly good buckling stability combined with sufficient effectiveness.

According to one embodiment, the Shore hardness of the inner tube is at most 50 shoreA. Conversely, the Shore hardness is particularly preferably greater than 30 shoreA. In particular, the Shore hardness of the inner tube is between 35 shoreA and 45 shoreA.

According to the second alternative, the coil is configured as a hollow coil, so that the coil has its own wall and the wall of the coil and the wall of the inner tube enclose a spiral-shaped cavity. The cavity is filled with air or another gas or has a vacuum. According to the third alternative, the coil is filled, i.e. it does not have a spiral-shaped cavity. The second alternative has the following advantage over the third alternative: With the same dimensions and the same inner tube and outer tube, the fluid guide unit is lighter than in the third embodiment. In addition, a hollow coil often leads to better thermal insulation than a filled coil. This is because a gas, in particular air, in the spirally circulating hollow space generally has a lower thermal conductivity than the material from which the wall of the coil is made.

Preferably, the wall thickness of the hollow coil is greater than the wall thickness of the inner tube and preferably also greater than the wall thickness of the outer tube. The wall thickness of the coil is particularly preferably between 0.5 mm and 1.5 mm, especially between 0.7 mm and 1.2 mm. In one embodiment, the wall thickness of the inner tube and/or that of the outer tube is between 0.5 mm and 0.7 mm.

According to the invention, the spiral coil is mechanically connected to the inner tube and mechanically connected to the outer tube and has a coil height, also known as the spiral height or pitch. This coil height is also the distance between the outer surface of the inner tube and the inner surface of the outer tube. Preferably, the coil height is at least 0.3 cm, particularly preferably 0.5 cm, especially at least 1 cm. This configuration results in a wider spiral-shaped cavity than a smaller spiral coil height and provides better thermal insulation of the inner tube against the environment. Preferably, the spiral coil height is at most 3 cm, particularly preferably at most 2.5 cm, especially at most 2 cm. As a result, the fluid guide unit is sufficiently flexible in many cases and still has good buckling stability thanks to the features of the invention.

The invention also relates to a ventilation arrangement which is configured to artificially ventilate a patient. The ventilation arrangement comprises a patient-side coupling unit. The patient-side coupling unit is configured to be arranged in and/or on the body of a patient during the artificial ventilation. The patient is therefore at least temporarily connected to a patient-side coupling unit or can be connected to such a unit. A tube and a breathing mask are two examples of a patient-side coupling unit. The ventilation arrangement also includes a ventilator. An anesthesia machine is a special case of a ventilator. A fluid guide unit connects the ventilator to the patient-side coupling unit. The entire fluid guide unit or at least one segment of the fluid guide unit is configured according to the invention.

The ventilator is configured to expel a breathable gas mixture. Preferably, the ventilator performs a sequence of ventilation strokes and expels in each ventilation stroke a respective quantity of the breathable gas mixture. The breathable gas mixture comprises oxygen and, in one application, additionally at least one anesthetic agent and/or a drug. The ventilation arrangement is configured to guide a gas mixture, which is expelled from the ventilator, through the fluid guide unit according to the invention to the patient-side coupling unit. The embodiments and advantages described above with reference to the fluid guide unit according to the invention also apply to the ventilation arrangement with the fluid guide unit according to the invention.

In one embodiment, a ventilation circuit is established between the ventilator and the patient-side coupling unit. The breathable gas mixture expelled by the ventilator is passed through an inspiration (inspiratory) fluid guide unit to the patient-side coupling unit. The gas mixture exhaled by the patient is passed back to the ventilator through an expiration (expiratory) fluid guide unit. A segment of the inspiration fluid guide unit and/or a segment of the expiration fluid guide unit are configured according to the invention, optionally the entire inspiration fluid guide unit and/or the entire expiration fluid guide unit.

It is also possible for a fluid guide unit according to the invention to have an inner tube and a further inner tube, with the further inner tube being fitted inside the inner tube. The fluid guide unit according to this embodiment thus comprises a two-lumen tube. One inner tube is used for inspiration and the other inner tube is used for expiration. The outer tube surrounds both inner tubes. The coil is connected to the outer tube and to the outer inner tube. This configuration saves space compared to a configuration in which two separate fluid guide units are used. In addition, the release of thermal energy to the environment is further reduced. Preferably, the inner, inner tube guides the breathable gas mixture to the patient-side coupling unit, and the outer, inner tube guides the exhaled gas mixture away from the patient-side coupling unit. In one embodiment, the entire two-lumen tube or at least one segment of this two-lumen tube is configured according to the invention.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an exemplary ventilation arrangement with a ventilation circuit in which the invention is used;

FIG. 2 is a cross-sectional view through a segment of the line according to the invention with a hollow coil;

FIG. 3 is a perspective cross-sectional view through the line according to the invention;

FIG. 4 is a cross-sectional view through a segment of the line according to the invention with a filled coil;

FIG. 5 is a schematic illustration of how Shore hardness is measured;

FIG. 6 is a schematic sectional view of a line without an outer tube and with insufficient buckling stability;

FIG. 7 is a schematic sectional view of a line with an outer tube and insufficient buckling stability;

FIG. 8 is a schematic sectional view of a line without an outer tube and with sufficient buckling stability; and

FIG. 9 is a schematic sectional view of a line with an outer tube and with sufficient buckling stability.

### DESCRIPTION OF PREFERRED EMBODIMENTS

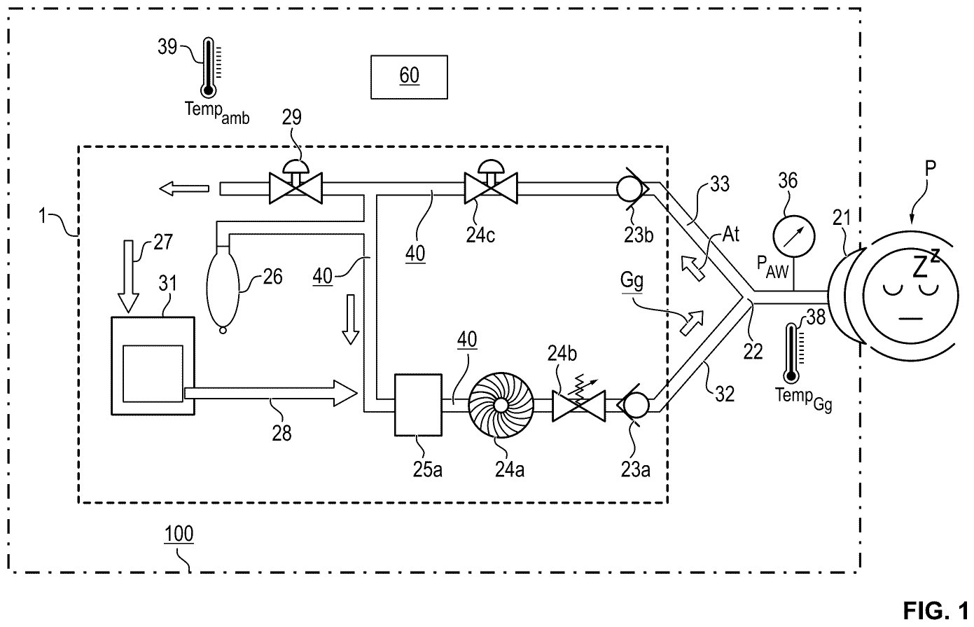

Referring to the drawings, the invention can be used in a ventilation arrangement 100. In the embodiment example, the ventilation arrangement 100 provides a ventilation circuit 40, which is shown schematically in FIG. 1. In this application, a patient P is artificially ventilated. Only the face of patient P is shown schematically. A ventilator 1, shown schematically, maintains a ventilation circuit 40. A patient-side coupling unit 21, shown only schematically, is arranged in and/or on the body of the patient P and connects the patient P to the ventilation circuit 40. In the embodiment example, an endotracheal tube of the patient-side coupling unit 21 bridges the nasal and pharyngeal cavity of the patient P. The patient-side coupling unit 21 may comprise a mouthpiece and/or a breathing mask.

In the example shown, the ventilator 1 is configured as an anesthesia device. The patient P is supplied with a breathable gas mixture Gg. For this purpose, the ventilator 1 performs a sequence of ventilation strokes and expels a quantity of the gas mixture Gg in each ventilation stroke. This gas mixture Gg comprises oxygen, at least one anesthetic agent and optionally a carrier gas for the anesthetic agent and optionally at least one gaseous drug.

An anesthetic dispenser (anesthetic dosing unit) 31 generates a fluid flow 28 of vaporous anesthetic, the fluid flow 28 being directed to the ventilation circuit 40. The gas mixture Gg, which is delivered to the patient P, is generated using this fluid flow 28. The anesthetic dispenser 31 generates the fluid flow 28 by feeding a vaporous anesthetic agent into a fluid flow 27 with a carrier gas, for example by injection or vaporization or evaporation. The carrier gas consists of breathing air or pure oxygen (O₂) and optionally also includes nitrous oxide (N₂O).

The breathable gas mixture Gg that reaches patient P has a temperature Temp_{Gg} of about 37 degrees C. and a relative humidity of about 75% or higher. The temperature Temp_{Gg} corresponds approximately to the temperature in the lungs of patient P, and the high humidity prevents the mucous membranes of patient P's airways from drying out. Therefore, the ventilator 1 comprises a humidifier 25a for the gas mixture Gg. An optional temperature sensor 39 measures the ambient temperature Temp_{amb}, i.e. the temperature in the environment of the ventilation circuit 40.

Note: The following refers to a sensor measuring a physical quantity. This means the following: The sensor directly measures the physical quantity or another quantity that correlates with the physical quantity to be measured, i.e. that is an indicator of the physical quantity to be measured.

The patient-side coupling unit 21 is connected by a tube to the base part of a Y-piece 22, which Y-piece 22 is preferably made of a rigid material. One leg of the Y-piece 22 is connected to an inspiration (inspiratory) line 32 for inhalation (inspiration), the other leg to an expiration (expiratory) line 33 for exhalation (expiration). Both lines 32, 33 and the Y-piece 22 are located outside of a housing of the ventilator 1. The rest of the ventilation circuit 40 is located in the housing. The inspiration line 32 is connected to a connection (port) in the housing of the ventilator 1 and guides the expelled breathable gas mixture Gg to the patient-side coupling unit 21. The entire inspiration line 32 or at least one segment of the inspiration line 32 functions as the or a flexible fluid guide unit within the meaning of the invention. The expiratory line 33 is connected to a further connection (port) in the housing and guides the gas mixture At exhaled by the patient P back to the ventilator 1. Preferably, the entire expiration line 33 or at least one segment of the expiration line 33 is also configured according to the invention. It is also possible that a two-lumen tube with two inner tubes acts as both the inspiration line 32 and the expiration line 33 and that one segment of each of the two lines 32, 33 is configured according to the invention. An inner, inner tube acts as the inspiration line 32 and an outer, inner tube acts as the expiration line.

A fluid delivery unit 24a in the form of a pump or a blower generates a permanent flow of the breathable gas mixture Gg through the inspiration line 32 to the Y-piece 22 and the patient-side coupling unit 21 and thus to the patient P. The gas flow in the ventilation circuit 40 is kept going by the fluid delivery unit 24a and optionally by a breathing bag 26, which can be operated manually.

Depending on its position, a controllable proportional valve 24b allows the gas flow generated by the pump 24a to pass through or blocks the generated gas flow, thereby contributing to the generation of the individual ventilation strokes and determining the respective duration and the amplitudes and frequencies of these ventilation strokes.

A check valve (non-return valve) 23a allows a gas flow in the inspiration line 32 to pass through to the Y-piece 22 and blocks a gas flow in the opposite direction. A check valve 23b allows a gas flow in the expiration line 33 to pass away from the Y-piece 22 and blocks a gas flow in the opposite direction. In one embodiment, the two non-return valves 23a and 23b are simple mechanical valves that open or close depending on which side has the greater pressure.

A pressure sensor 36 measures the current pressure in the ventilation circuit 40, for example the ventilation pressure applied to the patient P (airway pressure, Paw), in one embodiment as a differential pressure relative to the ambient pressure Pamb. In one embodiment, the measuring position of the pressure sensor 36 is located on the tube between the patient-side coupling unit 21 on the one hand and the Y-piece 22 on the other.

A PEEP valve 24c (PEEP= “positive end-expiratory pressure”) ensures that a sufficiently high air pressure is maintained in the lungs of patient P even at the end of exhalation or if the ventilation circuit 40 is briefly opened or interrupted. This reduces the risk of patient P's lungs collapsing due to insufficient pressure.

A pressure relief valve 29 is able to relieve excess pressure in the ventilation circuit 40 by causing gas to escape from the ventilation circuit 40. Preferably, the released gas is discharged into a transport line for anesthetic gas and reaches a conditioner (purifier) not shown.

In the embodiment example, the invention is used at least for the inspiration line 32. The inspiration line 32 is flexible at least in one segment outside the ventilator 1 in order to adapt it to the position of the ventilator 1 relative to the patient-side coupling unit 21 and thus to the patient P. Ideally, the inspiration line 32 is flexible along its entire length, i.e. from the housing of the ventilator 1 to the Y-piece 22. In a preferred embodiment, what is stated below for the inspiration line 32 applies accordingly to the expiration line 33.

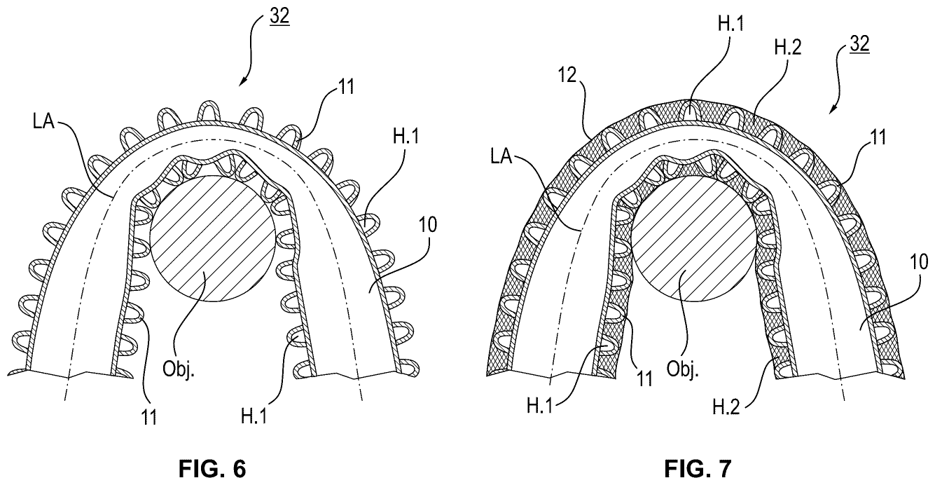

In particular, the inspiration line 32 should meet the following requirements:
It is possible that the inspiration line 32 is routed around a rigid object and thereby kinked (bent, buckled), see FIG. 6 to FIG. 9. This situation is often unavoidable. Nevertheless, the flow of the breathable gas mixture Gg to the patient P must not be significantly reduced so as not to jeopardize artificial ventilation.
One objective is to ensure that the temperature Temp_{Gg} of the breathable gas mixture Gg flowing through the inspiration line 32 is approximately 37 degrees C., i.e. the same as the temperature in the patient's lungs P within a tolerance.
As a rule, the ambient temperature Temp_{amb} is lower than the required temperature Temp_{Gg} of the breathable gas mixture Gg in the inspiration line 32. Therefore, not too much heat energy should be released into the environment.

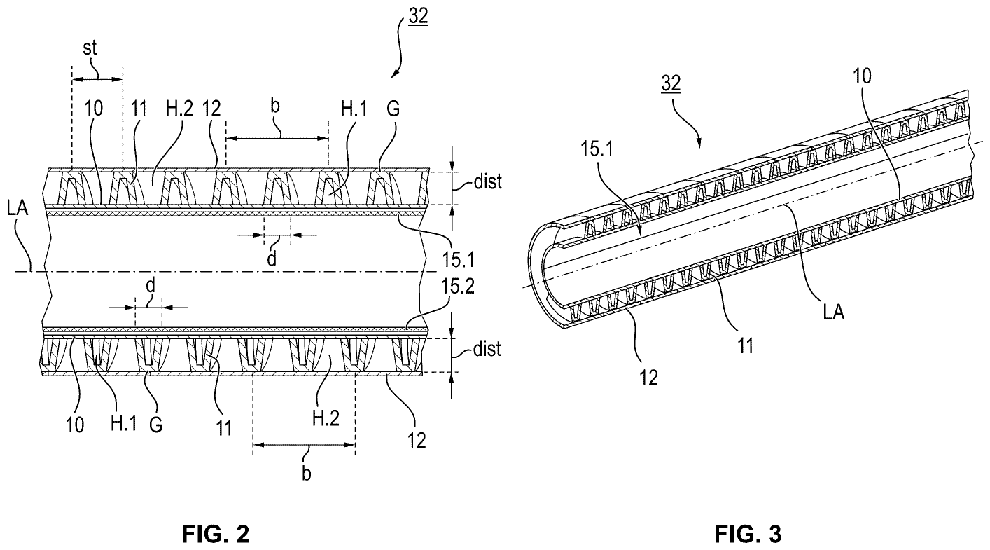

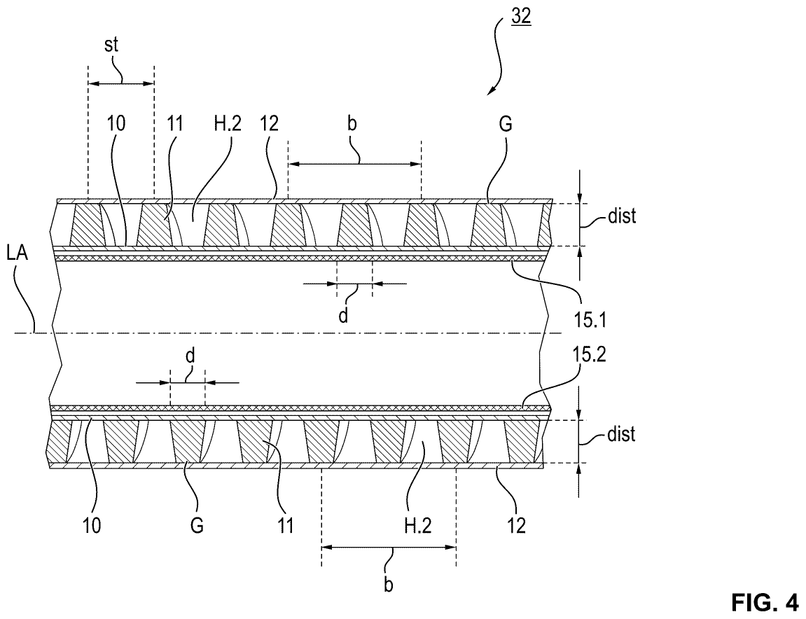

FIG. 2, FIG. 3, and FIG. 4 show two cross-sections through the inspiration line 32. The inspiration line 32 comprises a smooth, flexible inner tube 10 which conducts the gas mixture Gg from the ventilator 1 to the Y-piece 22.

As a heating element, the inspiration line 32 of the embodiment example comprises two parallel heating wires 15.1 and 15.2. In the implementation shown, the two heating wires 15.1 and 15.2 are applied internally to the inner surface of the inner tube 10 and therefore come into thermal contact with the gas mixture Gg flowing through the inner tube 10. As a result, the heat of the current-carrying heating wires 15.1 and 15.2 is transferred well to the gas mixture Gg. Conversely, the gas mixture Gg cools the heating wires 15.1 and 15.2. It is also possible that at least one heating wire 15.1, 15.2 is embedded in the inner tube 10 or is guided in a spiral around the inner tube 10.

A target range for the temperature Temp_{Gg} of the breathable gas mixture Gg is specified. A control unit 60, shown schematically in FIG. 1, causes an electrical voltage to be applied to the heating wires 15.1 and 15.2 in such a way that ideally the actual temperature Temp_{Gg} of the heated gas mixture Gg is always within this target range. The electrical voltage that the control unit 60 applies to the heating wires 15.1, 15.2 preferably depends on the measured actual temperature Temp_{Gg} and/or on the measured ambient temperature Temp_{amb}.

In one embodiment, the control unit 60 performs an open-loop control. In another embodiment, the control unit 60 performs a closed-loop control of the actual temperature Temp_{Gg} of the breathable gas mixture Gg flowing through the inspiration line 32. In this other embodiment, an optional temperature sensor 38 measures the actual temperature Temp_{Gg} of the breathable gas mixture Gg flowing through the inspiration line 32. The measuring position of the temperature sensor 38 can be at the Y-piece 22 or at the inspiration line 32 or at the tube between the Y-piece 22 and the inspiration line 32. The control objective (control gain) is to ensure that the measured actual temperature Temp_{Gg} of the breathable gas mixture Gg remains within a specified target temperature range. If the control deviation is too large, the control unit 60 causes the temperature of the heating wires 15.1, 15.2 to be increased or reduced.

A coil 11, i.e. a spiral, is applied to the smooth inner tube 10. In one implementation, this spiral-shaped coil 11 is hollow on the inside, cf. FIG. 2 and FIG. 3. In an alternative implementation the coil 11 is filled (non-hollow), cf. FIG. 4. A line (conduit) with a hollow inner coil is easier to bend and form into a desired shape than a line with a completely filled coil. A hollow coil is also lighter than a completely filled coil. Furthermore, thanks to the hollow configuration, the thermal insulation between the inspiration line 32 and the environment is improved.

The following parameters of the coil 11 are shown in FIG. 2 and FIG. 4:
the distance dist between the outer wall of the inner tube 10 and the inner wall of the outer tube 12, whereby the distance dist is also the coil height (pitch) of the coil 11,
the gradient (spiral coil spacing distance between coil ridges) st of coil 11 and
the thickness d of the coil 11, i.e. the maximum dimension of the coil 11 in a direction parallel to the longitudinal axis LA of the inner tube 10.

As already mentioned, the temperature Temp_{Gg} of the breathable gas mixture Gg, which is passed through the inner tube 10, is generally higher than the ambient temperature Temp_{amb}. Therefore, heat energy is inevitably released into the environment. As is well known, the larger the surface area is, the greater is the heat energy that is transferred over a surface area, given a constant temperature difference and a constant material. A coil has a large surface area.

Therefore, according to the invention, the surface of the inspiration line 32, which is in contact with the environment, has been reduced compared to a corrugated tube. For this purpose, a smooth tubular outer tube 12 is placed around the coil 11. Preferably, the smooth inner tube 10 is concentrically arranged in the smooth tubular outer tube 12, and the coil 11 bridges the constant distance between the inner tube 10 and the outer tube 12. In other words, the coil height (pitch) dist of the coil 11 is exactly as great as the distance between the inner tube 10 and the outer tube 12. A spiral-shaped circumferential ridge G of the coil 11 is mechanically connected to the inner surface of the outer tube 12.

According to the implementation shown in FIG. 2 and FIG. 3, a spiral-shaped cavity H.1 is therefore formed inside the coil 11, which is bounded by the wall of the coil 11 and by the wall of the inner tube 10. According to FIG. 2 to FIG. 4, between the two tubes 10 and 12 a further spiral-shaped cavity H.2 is provided, which cavity H.2 is bounded by the hollow or filled coil 11 and the two tubes 10 and 12.

In many cases, the surface area of the inspiration line 32 that comes into contact with the environment in an embodiment with the smooth outer tube 12 has less than 60% of the surface area that occurs in a conceivable embodiment in which the coil 11 comes into contact with the environment.

The two tubes 10 and 12 and the coil 11 are flexible components. The inventors have examined and compared several potential configurations of the inspiration line 32 in internal tests. One criterion for evaluating different configurations is buckling stability. FIG. 6 and FIG. 7 show two potential embodiments that lead to insufficient buckling stability. In both embodiments, the inspiration line 32 is guided around a rigid object in the form of a rod. The curved longitudinal axis LA of the inspiration line 32 lies in the drawing planes of FIG. 6 and FIG. 7, and the straight longitudinal axis of the rod-shaped object Obj is perpendicular to the longitudinal axis LA and thus perpendicular to the drawing planes of FIG. 6 and FIG. 7. A similar situation can also occur in practice. As can be seen, the cross-section available for the flow of the breathable gas mixture Gg and thus also the volume flow, i.e. the volume per unit time that can flow through the inspiration line 32, are considerably reduced. One cause is as follows: The inner tube 10 is compressed on the inside, i.e. on the surface facing the object Obj. This situation can endanger the artificially ventilated patient P and should therefore be avoided.

In the potential embodiment shown in FIG. 6, the outer tube 12 is left out and the coil 11 is in thermal contact with the environment. In the potential embodiment shown in FIG. 7, the outer tube 12 is attached to the coil 11.

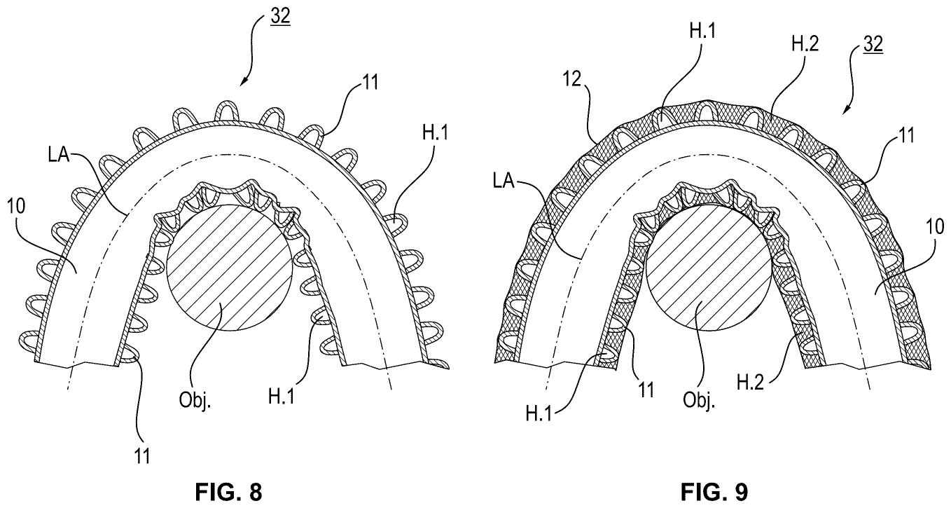

In the designs shown in FIG. 6 and FIG. 7, the inner tube 10, the coil 11 and the outer tube 12 have the same Shore hardness. A modification has now been tested. The Shore hardness of the coil 11 is at least 10 shoreA greater than the Shore hardness of the inner tube 10 and at least 10 shoreA greater than the Shore hardness of the outer tube 12. FIG. 8 and FIG. 9 show the results from the same viewing direction as FIG. 6 and FIG. 7. The same reference symbols (characters) have the same meanings. It can be seen that the inside of the inner tube 10 is relatively little compressed. The available cross-section and thus the achievable volume flow are significantly less reduced than in the embodiments shown in FIG. 6 and FIG. 7.

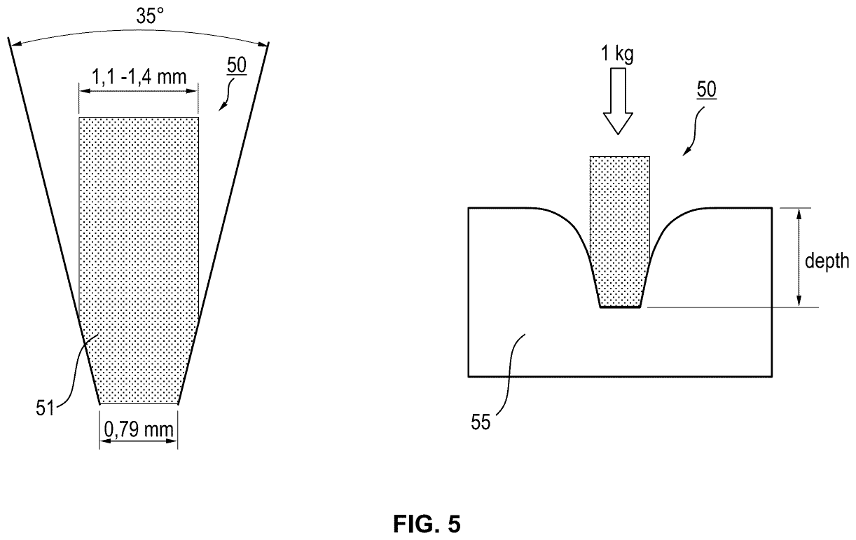

Two key parameters that influence the buckling stability of a tubular flexible object are the Shore hardness and the wall thickness. FIG. 5 illustrates a standardized method for measuring the Shore hardness shoreA. A rigid test body 50 with a mass of 1 kg comprises a truncated cone 51. The smaller end face of the truncated cone 51 has a diameter of 0.79 mm, the larger end face has a diameter of between 1.1 mm and 1.4 mm. The lateral surface of the truncated cone 51 is at an angle of 35 degrees to the two end faces of the truncated cone 51. The test body 50 is positioned above an object 55 to be tested. The smaller end face of the truncated cone 51 points towards the object 55. The test body 50 is moved vertically downwards by its own weight and penetrates the object 55 for 15 seconds. The penetration depth is measured and mapped linearly on a scale from 0 shoreA to 100 shoreA. Preferably, the penetration depth is measured several times and the arithmetic mean or the median is calculated. A penetration depth of at least 2.5 mm corresponds to a Shore hardness of 0 shoreA, a penetration depth of 0 mm corresponds to a Shore hardness of 100 shoreA.

In the embodiment example, the coil 11 has a Shore hardness of between 70 shoreA and 90 shoreA, in particular a Shore hardness of between 75 shoreA and 85 shoreA. Both the inner tube 10 and the outer tube 12 each have a Shore hardness of between 30 shoreA and 50 shoreA, preferably between 35 shoreA and 45 shoreA. It is possible that the inner tube 10 has the same Shore hardness as the outer tube 12 or a greater Shore hardness than the outer tube 12. The inner tube 10 and the outer tube 12 each have a wall thickness of between 0.5 mm and 0.7 mm. The wall thickness of the hollow coil 11 is between 0.7 mm and 1.2 mm. There is a distance between the inner tube 10 and the outer tube 12, which is preferably constant over the entire length of the inspiration line 32 and is between 3.0 mm and 4.2 mm, preferably between 3.3 mm and 3.7 mm. The coil 11 completely bridges this distance and therefore has a coil height that is equal to the distance.

A preferred method of manufacturing the inspiration line 32 comprises the following steps:
The inner tube 10 is provided.
The coil 11 is applied to the outside of the inner tube 10. Preferably, the coil 11 is applied by vulcanization.
A web (band) is wound around the coil 11 in such a way that the center axis of the web is parallel to the spiral ridge G of the coil 11. This web forms the outer tube 12. Preferably, the web is joined to the coil 11 by vulcanization.
Preferably, the web is so wide (broad) that a section that runs from one edge to the other edge of the web and is perpendicular to the center axis of the web covers three ridges. The width b of this web is shown schematically in FIG. 2 and FIG. 4.

It is possible that the expiration line 33 is configured in the same way as the inspiration line 32.

Preferably, an elastomer is used as the material for each of the two tubes 10, 12 and for the coil 11.

While specific embodiments of the invention have been shown and described in detail to illustrate the application of the principles of the invention, it will be understood that the invention may be embodied otherwise without departing from such principles.

List of reference characters

| | |
|---|---|
| 1 | Ventilator, configured as an anesthesia device, performs a sequence of |
| | ventilation strokes, expels in each ventilation stroke a respective quantity |
| | of the breathable gas mixture Gg, maintains a gas flow in the ventilation |
| | circuit 40 |
| 10 | Smooth inner tube of the inspiration line 32, conducts the breathable gas |
| | mixture Gg from the ventilator 1 to the Y-piece 22, surrounded by the coil |
| | 11, accommodates the heating wires 15.1, 15.2 |
| 11 | Spiral coil on the smooth inner tube 10, is hollow or filled |
| | Smooth tubular outer tube on the coil 11 |
| 15.1, | Electrically conductive heating wires, which heat the gas mixture Gg in |
| 15.2 | the inner tube 10, arranged in the inner tube 10 |
| 21 | Patient-side coupling unit, connected to the patient P and to the Y-piece |
| | 22 |
| 22 | Y-piece, connects the two lines 32 and 33 with each other and with the |
| | patient-side coupling unit 21 |
| 23a | Non-return valve in the inspiration line 32 |
| 23b | Non-return valve in the expiration line 33 |
| 24a | Fluid delivery unit in the form of a blower or a pump, maintains the gas |
| | flow in the ventilation circuit 40 |
| 24b | Controllable proportional valve, determines the amplitudes and |
| | frequencies of the ventilation strokes |
| 24c | PEEP valve, prevents the patient's lungs from collapsing P |
| 25a | Humidifier, ensures that the breathable gas mixture Gg in the inspiration |
| | line 32 has a relative humidity of at least 75% |
| 26 | Breathing bag with which the ventilation circuit 40 can be maintained |
| | manually |
| 27 | Fluid flow of fresh gas, is fed to the anesthetic dispenser 31 |
| 28 | Fluid flow with vaporous anesthetic agent is generated from the fluid |
| | flow 27 by the anesthetic agent dispenser 31 |
| 29 | Pressure relief valve, can relieve excess pressure in the ventilation circuit |
| | 40 |
| 31 | Anesthetic dispenser, generates the fluid flow 28 |
| 32 | Inspiration line for inhalation (inspiration) |
| 33 | Expiration line for exhalation (expiration) |
| 36 | Pressure sensor, measures the current pressure in the ventilation circuit 40 |
| 38 | Temperature sensor, measures the temperature Temp_{Gg} of the breathable |
| | gas mixture Gg flowing to the patient-side coupling unit 21 |
| 39 | Temperature sensor, measures the ambient temperature Temp_{amb} |
| 40 | Ventilation circuit between the ventilator 1 and the patient P, comprises |
| | the lines 32 and 33 |
| 50 | Test body (test body), comprising the truncated cone 51, is used to |
| | measure the Shore hardness of the object 55 |
| 51 | Truncated cone of the test body 50 |
| 55 | Object whose Shore hardness is measured using the test body 50 |
| 60 | Control unit, receives signals from the sensors 36, 8 and 30, 39, controls, |
| | among other components, the anesthetic dispenser (anesthetic dosing |
| | unit) 31 and the valve 24c |
| 100 | Ventilation arrangement, comprising the ventilator 1, the control unit 60, |
| | the patient-side coupling unit 21, the Y-piece 22, the lines 32, 33 and the |
| | sensors 36, 38, 39 |
| At | Gas mixture exhaled by patient P flows through expiration line 33 to |
| | ventilator 1 |
| b | Width of a web that is guided around the coil 11 during production and |
| | forms the outer tube 12 |
| d | Thickness of the coil 11, i.e. maximum dimension parallel to the |
| | longitudinal axis LA |
| depth | Penetration depth of the test body 50 into the object 55, correlated with |
| | the Shore hardness |
| dist | Distance between the inner wall of the outer tube 12 and the outer wall of |
| | the inner tube 10, at the same time coil height (pitch) of the coil, i.e. |
| | dimension of the coil 11 in a direction perpendicular to the longitudinal |
| | axis LA |
| G | Ridge of the coil 11 |
| Gg | Breathable gas mixture that flows through the inspiration line 32 and |
| | whose temperature Temp_{Gg} is measured by the temperature sensor 38 |
| H.1 | Spiral-shaped cavity inside the coil 11 |
| H.2 | Spiral-shaped cavity between the tubes 10 and 12 and outside the coil 11 |
| LA | Longitudinal axis of the inspiration line 32 |
| Obj | Rigid object around which the inspiration line 32 is bent |
| P | Patient, connected to the patient-side coupling unit 21, is anesthetized and |
| | artificially ventilated |
| st | Pitch of the spiral coil 11 (distance between coil ridges) |
| Temp_{amb} | Ambient temperature, measured by temperature sensor 39 |
| Temp_{Gg} | Temperature of the breathable gas mixture Gg, measured by temperature |
| | sensor 38 |

What is claimed is:

1. A flexible fluid guide unit comprising:
an inner tube extending along a longitudinal axis and configured to guide a fluid, the inner tube having an inner tube Shore hardness;
an outer tube having an outer tube Shore hardness;
a spiral coil between the inner tube and the outer tube, wherein the spiral coil extends around the inner tube and is mechanically connected to both the inner tube and the outer tube, wherein the outer tube surrounds the inner tube and the spiral coil, wherein an outer surface of the outer tube is in thermal contact with an environment of the fluid guide unit and the outer surface of the outer tube is smaller than a sum of an outer surface of the spiral coil and a part of an outer surface of the inner tube which is not covered by the spiral coil, wherein the spiral coil has a spiral coil Shore hardness and wherein the spiral coil Shore hardness, measured in shoreA, is at least as high as the inner tube Shore hardness and at least as high as the outer tube Shore hardness; and
a heating element configured to heat a fluid guided by the inner tube,
wherein the spiral coil Shore hardness is at least 10 shoreA greater than the inner tube Shore hardness and the spiral coil Shore hardness is at least 10 shoreA greater than the outer tube Shore hardness, or
wherein the spiral coil is a hollow spiral coil and has a spiral coil wall which surrounds a spiral-shaped cavity and the spiral coil wall has a wall thickness which is at least twice as great as a wall thickness of the inner tube and at least twice as great as a wall thickness of the outer tube, or
wherein the spiral coil has the shape of a filled rod formed into a spiral, and a maximum dimension of the filled rod in a direction parallel to the longitudinal axis of the inner tube is at least four times as great as the wall thickness of the inner tube and at least four times as great as the wall thickness of the outer tube.

2. A fluid guide unit according to claim 1,
wherein the spiral coil Shore hardness is at least 20 ShoreA greater than the inner tube Shore hardness, and
wherein the spiral coil Shore hardness is at least 20 ShoreA greater than the outer tube Shore hardness.

3. A fluid guide unit according to claim 1,
wherein the spiral coil Shore hardness is at least 30 ShoreA greater than the inner tube Shore hardness, and
wherein the spiral coil Shore hardness is at least 30 ShoreA greater than the outer tube Shore hardness.

4. A fluid guide unit according to claim 1,
wherein the spiral coil Shore hardness is at least 70 shoreA,
wherein the inner tube Shore hardness is at most 50 shoreA, and
wherein the outer tube Shore hardness is at most 50 shoreA.

5. A fluid guide unit according to claim 1,
wherein the spiral coil Shore hardness is at least 70 shoreA and less than 90 shoreA,
wherein the inner tube Shore hardness is at most 50 shoreA and is greater than 30 shoreA, and
wherein the outer tube Shore hardness is at most 50 shoreA and greater than 30 shoreA.

6. A fluid guide unit according to claim 5,
wherein the spiral coil Shore hardness is less than 80 shoreA.

7. A fluid guide unit according to claim 1,
wherein the spiral coil Shore hardness between 75 shoreA and 85 shoreA,
wherein the inner tube Shore hardness between 35 shoreA and 45 shoreA, and
wherein the outer tube Shore hardness between 35 shoreA and 45 shoreA

8. A fluid guide unit according to claim 1,
wherein the hollow spiral coil has a shape of a hollow rod formed into a spiral, and
wherein the hollow rod formed into a spiral has a maximum dimension in the direction parallel to the longitudinal axis of the inner tube that is at least four times as great as the wall thickness of the inner tube and at least four times as great as the wall thickness of the outer tube.

9. A fluid guide unit according to claim 1,
wherein the hollow spiral coil has a shape of a hollow rod formed into a spiral,
wherein the wall thickness of the hollow spiral coil wall is between 0.5 mm and 1.5 mm, and
wherein the wall thickness of the wall thickness of the inner tube is smaller than the wall thickness of the spiral coil.

10. A fluid guide unit according to claim 9, wherein the wall thickness of the spiral coil wall is between 0.7 mm and 1.2 mm

11. A fluid guide unit according to claim 1, wherein the spiral coil has a coil height that is at least 0.3 cm.

12. A fluid guide unit according to claim 1, wherein the spiral coil has a coil height that is at least 0.5 cm.

13. A fluid guide unit according to claim 1, wherein the spiral coil has a coil height that is at least 1 cm.

14. A fluid guide unit according to claim 1, wherein the spiral coil has a coil height that is at most 3 cm.

15. A fluid guide unit according to claim 1, wherein the spiral coil has a coil height that is at most 2 cm.

16. A process comprising the steps of:
providing a ventilation arrangement for artificial ventilation of a patient, the ventilation arrangement comprising a ventilator, a patient-side coupling unit and a fluid guide unit;
providing the fluid guide unit so as to comprise: an inner tube extending along a longitudinal axis and configured to guide a fluid, the inner tube having an inner tube Shore hardness; an outer tube having an outer tube Shore hardness; a spiral coil between the inner tube and the outer tube, wherein the spiral coil extends around the inner tube and is mechanically connected to both the inner tube and the outer tube, wherein the outer tube surrounds the inner tube and the spiral coil, wherein an outer surface of the outer tube is in thermal contact with an environment of the fluid guide unit and the outer surface of the outer tube is smaller than a sum of an outer surface of the spiral coil and a part of an outer surface of the inner tube which is not covered by the spiral coil, wherein the spiral coil has a spiral coil Shore hardness and wherein the spiral coil Shore hardness, measured in shoreA, is at least as high as the inner tube Shore hardness and at least as high as the outer tube Shore hardness; and a heating element configured to heat a fluid guided by the inner tube,
wherein the spiral coil Shore hardness is at least 10 shoreA greater than the inner tube Shore hardness and the spiral coil Shore hardness is at least 10 shoreA greater than the outer tube Shore hardness, or
wherein the spiral coil is a hollow spiral coil and has a spiral coil wall which surrounds a spiral-shaped cavity and the spiral coil wall has a wall thickness which is at least twice as great as a wall thickness of the inner tube and at least twice as great as a wall thickness of the outer tube, or
wherein the spiral coil has the shape of a filled rod formed into a spiral, and a maximum dimension of the filled rod in a direction parallel to the longitudinal axis of the inner tube is at least four times as great as the wall thickness of the inner tube and at least four times as great as the wall thickness of the outer tube.

17. A ventilation arrangement for artificial ventilation of a patient, wherein the ventilation arrangement comprises:
a ventilator configured to expel a breathable gas mixture;
a patient-side coupling unit configured to be arranged in and/or on the body of a patient; and
a flexible fluid guide unit, wherein the ventilation arrangement is configured to guide a breathable gas mixture expelled by the ventilator through the fluid guide unit to the patient-side coupling unit, and wherein the entire fluid guide unit or at least one segment of the fluid guide unit is configured so as to comprise: an inner tube extending along a longitudinal axis and configured to guide a fluid, the inner tube having an inner tube Shore hardness; an outer tube having an outer tube Shore hardness; a spiral coil between the inner tube and the outer tube, wherein the spiral coil extends around the inner tube and is mechanically connected to both the inner tube and the outer tube, wherein the outer tube surrounds the inner tube and the spiral coil, wherein an outer surface of the outer tube is in thermal contact with an environment of the fluid guide unit and the outer surface of the outer tube is smaller than a sum of an outer surface of the spiral coil and a part of an outer surface of the inner tube which is not covered by the spiral coil, wherein the spiral coil has a spiral coil Shore hardness and wherein the spiral coil Shore hardness measured in shoreA, is at least as high as the inner tube Shore hardness and at least as high as the outer tube Shore hardness; and a heating element configured to heat the fluid guided by the inner tube,
wherein the spiral coil Shore hardness is at least 10 shoreA greater than the inner tube Shore hardness and the spiral coil Shore hardness is at least 10 shoreA greater than the outer tube Shore hardness, or
wherein the spiral coil is a hollow spiral coil and has a spiral coil wall which surrounds a spiral-shaped cavity and the spiral coil wall has a wall thickness which is at least twice as great as a wall thickness of the inner tube and at least twice as great as a wall thickness of the outer tube, or
wherein the spiral coil has the shape of a filled rod formed into a spiral, and a maximum dimension of the filled rod in a direction parallel to the longitudinal axis of the inner tube is at least four times as great as the wall thickness of the inner tube and at least four times as great as the wall thickness of the outer tube.

18. A ventilation arrangement according to claim 17, further comprising a further flexible fluid guide unit, wherein the ventilation arrangement is configured to guide a gas mixture exhaled by the patient through the further fluid guide unit to the ventilator, and the entire further fluid guide unit or at least one segment of the further fluid guide unit is configured so as to comprise: a further fluid guide inner tube extending along a longitudinal axis and configured to guide a fluid, the further fluid guide inner tube having a further inner tube Shore hardness; a further fluid guide outer tube having a further outer tube Shore hardness; a further fluid guide spiral coil between the further fluid guide inner tube and the further fluid guide outer tube, wherein the further fluid guide coil extends around the further fluid guide inner tube and is mechanically connected to both the further fluid guide inner tube and the further fluid guide outer tube, wherein the further fluid guide outer tube surrounds the further fluid guide inner tube and the further fluid guide spiral coil, wherein an outer surface of the further outer tube is in thermal contact with an environment of the further fluid guide unit and the outer surface of the further fluid guide unit outer tube is smaller than a sum of an outer surface of the further fluid guide unit spiral coil and a part of an outer surface of the further fluid guide unit inner tube which is not covered by the further fluid guide unit spiral coil, wherein the further fluid guide unit spiral coil has a further spiral coil Shore hardness and wherein the further spiral coil Shore hardness, measured in shoreA, is at least as high as the further inner tube Shore hardness and at least as high as the further outer tube Shore hardness; and a further fluid guide unit heating element configured to heat the fluid guided by the further fluid guide unit inner tube,
wherein the further spiral coil Shore hardness is at least 10 shoreA greater than the further inner tube Shore hardness and the further spiral coil Shore hardness is at least 10 shoreA greater than the further outer tube Shore hardness, or
wherein the further fluid guide unit spiral coil is a further fluid guide unit hollow spiral coil and has a further spiral coil wall which surrounds a further spiral-shaped cavity and the further spiral coil wall has a further spiral coil wall thickness which is at least twice as great as a further inner tube wall thickness of the further fluid guide unit inner tube and at least twice as great as a further outer tube wall thickness of the further fluid guide unit outer tube, or
wherein the further fluid guide unit spiral coil has the shape of a further fluid guide unit filled rod formed into a spiral, and a maximum dimension of the further fluid guide unit filled rod, in a direction parallel to the longitudinal axis of the further fluid guide unit inner tube, is at least four times as great as the further inner tube wall thickness and at least four times as great as the further outer tube wall thickness.

19. A ventilation arrangement according to claim 17,
wherein the spiral coil Shore hardness is at least 70 shoreA and less than 90 shoreA,
wherein the inner tube Shore hardness is at most 50 shoreA and is greater than 30 shoreA, and
wherein the outer tube Shore hardness is at most 50 shoreA and greater than 30 shoreA.

20. A ventilation arrangement according to claim 17,
wherein the hollow spiral coil has a shape of a hollow rod formed into a spiral, and wherein the hollow rod formed into a spiral has a maximum dimension in the direction parallel to the longitudinal axis of the inner tube that is at least four times as great as the wall thickness of the inner tube and at least four times as great as the wall thickness of the outer tube.
